(19) United States
(12) Patent Application Publication
(10) Pub. No.: US 2018/0228466 A1
(43) Pub. Date: Aug. 16, 2018

(54) Ultrasound Probe with Concave Shaped Head

(71) Applicant: Evelyn Kay Mann, Powhatan, VA (US)

(72) Inventor: Evelyn Kay Mann, Powhatan, VA (US)

(21) Appl. No.: 15/658,878

(22) Filed: Jul. 25, 2017

Related U.S. Application Data
(60) Provisional application No. 62/499,983, filed on Feb. 10, 2017.

(51) Int. Cl. A61B 8/00

(52) U.S. Cl. CPC A61B 8/4455

(57) ABSTRACT

An ultrasound probe with a generally concave shaped head and a handle proceeding away from the generally concave shaped head. The probe can be concave in one or two axes. Such a device is easier to point and more comfortable for the patient when used by a technician on the generally convex shape of an animal arm, leg, or abdomen.

### CROSS-REFERENCE TO RELATED APPLICATIONS

This application takes benefit of U.S. Provisional App. No. 62/499,983 filed Feb. 10, 2017 which is hereby included in its entirety by reference.

### FIELD OF THE INVENTION

The present invention relates to an ultrasound probe with a concave shaped head. Such a device is easier to point and more comfortable when used by a technician on the generally convex shape of an animal arm, leg, or abdomen.

### BACKGROUND OF THE INVENTION

Ultrasound devices are used often in the practice of medicine. Whether studying pregnancies, torn joints, sprained discs, or virtually any other structural manifestation of the animal condition, an ultrasound device is an excellent observational tool that is relatively easy to use and relatively painless to use on the recipient.

Ultrasound devices have their drawbacks however. One drawback is the ultrasound transmission gel which is necessary to acoustically couple the ultrasound probe head to the skin of the patient. A second drawback is that ultrasound probes are universally flat or convex in shape. This shape does not intimately conform to the typical shape of animals, to wit, slightly too markedly convex.

What is needed then is an ultrasound probe physically constructed such that the contact surface is concave to match the typical shape of an animal limb or form.

### SUMMARY OF THE INVENTION

It is an object of the present invention to provide an improved ultrasound probe that possesses a concave contact surface for use on generally convex animal surfaces. For example, the abdomen of a pregnant woman is markedly convex. An ultrasound probe that possesses a concave contact surface would therefore physically conform to the abdomen and would be physically less offensive to the patient when used.

The present invention is used the same way existing examples in the prior art are used, specifically: First, the operator places ultrasound transmission gel on the part of the body where an ultrasound survey is desired. Next, the user contacts the concave contact surface to the generally convex surface of the body. Next, the operator slides the ultrasound probe laterally and fore and aft on the surface of the body to fix the proper place for study. Finally, the operator rotates the ultrasound probe in contact with the skin of the patients along its widest axis to change the focal point of the ultrasound probe to acoustically scan the studied region.

### BRIEF DESCRIPTION OF THE DRAWINGS

FIG. 1 is a perspective view of the exterior of an improved ultrasound probe showing the concave contact head.

FIG. 2 is a plan view of the exterior of an improved ultrasound probe showing the concave contact head.

### DETAILED DESCRIPTION OF THE EMBODIMENTS

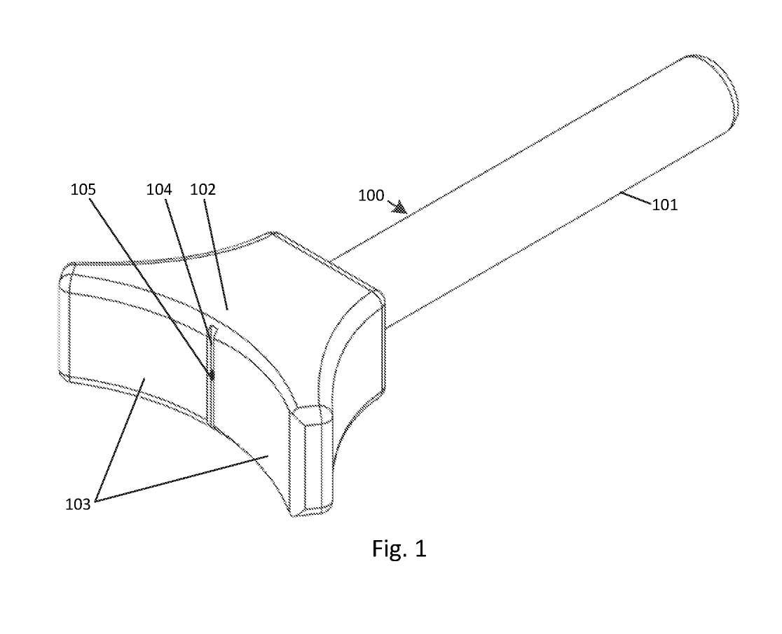

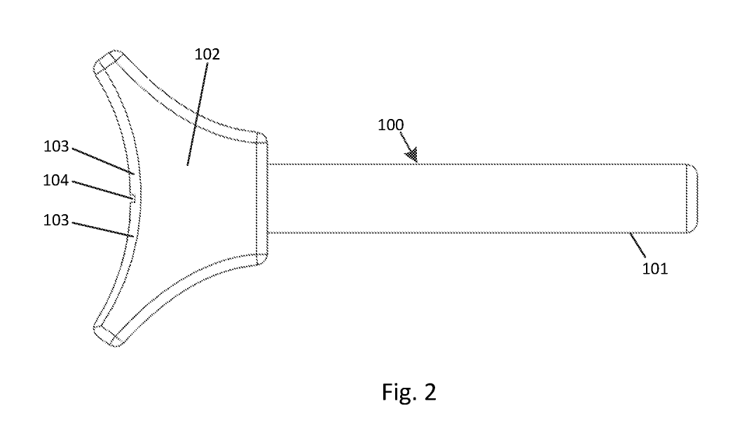

In the first embodiment, pictured in FIGS. 1 and 2, the present invention is an improved ultrasound probe 100 that possesses concave contact surface 103 for use on generally convex animal surfaces. Ultrasound probe 100 is equipped with handle or holding structure 101 permanently attached to sensing head 102. Sensing head 102 has a concave contact surface 103.

Ultrasound probe 100 is shown with a linear handle or holding structure 101, but it will be readily apparent to those having skill in the art that any shape suitable for being held in the hand such that sensing head 102 may be held in intimate contact with the animal surface is suitable.

Sensing head 102 is shown with convex contact surface 103 and curvilinear back surfaces that connect to handle or holding structure 101. It will be readily apparent to those having skill in the art that any shape suitable for physically containing the electronic components of sensing head 102 may be used for the back surfaces.

Sensing head 102 contains a concave ultrasonic transducer or an ultrasonic transmitter and receiver, but it will be readily obvious that sensing head 102 may contain alternative components, including printed, painted, or molded markings 104, heating elements, light emitting diodes (LEDs), laser diodes 105, and the like.

Ultrasonic transducers are typically constructed of lead zirconate titanate (PZT-PT) or relaxor single crystals (PMN-PT) because of the relatively high conversion efficiency of transducers constructed of such materials. Similarly, it is well known to those having skill in the art that materials exhibiting a non-piezoelectric principle such as magnetostriction may be used to construct ultrasonic transducer.

Concave contact surface 103 lies distal to a concave acoustic matching layer which lies distal to concave piezoelectric element which is distally bonded to a concave acoustic backing plate.

Those having skill in the art will recognize that a concave acoustic backing plate constructed of fiberglass or poly-vinyl chloride (PVC) on brass are examples of the multitude of substances from which such backing plates may be constructed.

Those having skill in the art will also recognize that a concave acoustic matching layer constructed of epoxy, sol-gel SiO₂, a TiO₂ nano-structured material, or other plastic materials are common examples of substances used to construct such acoustic matching layers.

Concave contact surface 103 serves as the acoustic lens of ultrasound probe 100 and is typically constructed of acrylic plastic (PMMA). Those having skill in the art will recognize that there are multiple alternate materials and conformal coatings (such as Stycast 1090SI) that are also suitable.

The present invention is used similarly to existing examples in the prior art, specifically: First, the operator places ultrasound transmission gel on the general part of the body that an ultrasound survey is desired. Next, the user applies concave contact surface 103 of ultrasound probe 100 to the generally convex surface of the body. Next, the operator slides ultrasound probe 100 laterally and fore and aft across the surface of the body to determine the proper place for study. Finally, the operator rotates ultrasound probe 100 along its widest axis (the long axis of concave contact surface 103) to change the focal point of the ultrasound probe to scan the studied region.

While the invention has been described in connection with what are considered to be the most practical and preferred embodiments, it is to be understood that the invention is not to be limited to the disclosed embodiments, but on the contrary is intended to cover various modifications and equivalent arrangements included within the spirit and scope of the disclosure.

What is claimed is:

1. An improved ultrasound probe, comprising:
a) a handle; and
b) a sensing head with a generally concave contact surface wherein the concavity is aligned along the wide axis of the sensing head.

2. An improved ultrasound probe of claim 1 wherein a generally concave contact surface is also aligned along the narrow axis of the sensing head.

3. An improved ultrasound probe of claim 1 wherein the exposed sensing head is comprised of a conformal coating.

4. An improved ultrasound probe of claim 1 wherein the exposed sensing head is comprised of acrylic plastic.

5. An improved ultrasound probe of claim 1 wherein the exposed sensing head has a groove.

6. An improved ultrasound probe of claim 1 wherein the exposed sensing head has a light emitting diode.

7. An improved ultrasound probe of claim 1 wherein the exposed sensing head has a laser diode.
